(19) United States
(12) Patent Application Publication
(10) Pub. No.: US 2014/0311488 A1
(43) Pub. Date: Oct. 23, 2014

(54) Assembly for an Inhalation Device, Use of an Organizing Member, and Inhalation Device

(71) Applicant: Stefan Mayer, Freiburg Im Breisgau (DE)

(72) Inventor: Stefan Mayer, Freiburg Im Breisgau (DE)

(73) Assignee: SANOFI SA, Vernier (CH)

(21) Appl. No.: 14/361,189

(22) PCT Filed: Nov. 14, 2012
(86) PCT No.: PCT/EP2012/072626

§ 371 Date: May 28, 2014

(30) Foreign Application Priority Data
Nov. 30, 2011 (EP) 11191386.9

(51) Int. Cl. A61M 15/00

(52) U.S. Cl. CPC A61M 15/0006; A61M 15/0065
USPC 128/203.15; 128/203.12

(57) ABSTRACT

An assembly for the inhalation of a powdery substance is described, wherein the assembly comprises a storage chamber configured to hold a plurality of doses of the substance and an organizing member rotatably arranged within the storage chamber for agitating the substance when the organizing member is rotated with respect to the storage chamber. The organizing member comprises a main body and a cut-out, wherein the main body comprises a solid section of the organizing member, and wherein the angular dimension of the cut-out is smaller than the angular dimension of the main body. Furthermore, the use of an organizing member for agitating a substance is provided. Furthermore, an inhalation device for the inhalation of a powdery substance is described.

### CROSS REFERENCE TO RELATED APPLICATIONS

The present application is a U.S. National Phase Application pursuant to 35 U.S.C. §371 of International Application No. PCT/EP2012/072626 filed Nov. 14, 2012, which claims priority to European Patent Application No. 11191386.9 filed Nov. 30, 2011. The entire disclosure contents of these applications are herewith incorporated by reference into the present application.

### FIELD OF INVENTION

This disclosure relates to an assembly for an inhalation device, to an inhalation device comprising such an assembly and to the use of an organizing member for agitating a substance retained in a storage chamber of such an inhalation device.

An inhalation device is described in document WO 2009/065707 A1, for example.

### BACKGROUND

It is an object of the present disclosure to provide an assembly for an improved inhalation device, e.g. a more efficient inhalation device. Furthermore, it is an object of the present disclosure to provide an improved inhalation device. Furthermore, it is an object of the present disclosure to provide the use of an organizing member to facilitate provision of an improved inhalation device.

This object may, inter alia, be achieved by the subject matter of the independent claims. Advantageous embodiments and refinements are the subject matter of the dependent claims. However, further advantageous concepts may be disclosed herein besides the ones which are claimed.

### SUMMARY

One aspect relates to an assembly for an inhalation device, in particular an assembly for the inhalation of a, e.g. powdery, substance. The assembly comprises a storage chamber. The storage chamber may be configured to hold at least one, preferably a plurality of doses of the substance. The assembly may further comprise an organizing member. The organizing member may be rotatably arranged within the storage chamber. The organizing member may be adapted and arranged to rotate about the main longitudinal axis of the storage chamber. The organizing member may be adapted and arranged for agitating the substance when the organizing member is rotated with respect to the storage chamber. The organizing member may comprise a main body. The main body may comprise a solid section of the organizing member. The organizing member, in particular the main body of the organizing member, may comprise a cut-out. The main body may comprise an angular dimension. The cut-out may comprise an angular dimension. The angular dimension of the cut-out may be smaller than the angular dimension of the main body.

The organizing member, in particular the main body of the organizing member, may reduce the volume which is available for the substance within the storage chamber. Powder may only be received within the cut-out of the organizing member, for example, which has a smaller angular dimension than the main body. Due to this specific shape of the organizing member, a residual volume which remains in the storage chamber after the last dose of the substance was delivered may be minimized. This may help to facilitate provision of a cost-efficient inhalation device.

In addition to that, the organizing member may, particularly due to its asymmetric shape which is given by the cut-out, agitate the substance in the storage chamber. In other words, the cut-out may have the function of a symmetry breaker. In particular, when the organizing member is rotated, powder, in particular powder bridges, which may be arranged close to an inner wall of the storage chamber may be received in the cut-out and may, thus, be broken up. The powder bridges may be arranged along the inner wall of the storage chamber. The powder bridges may prevent a spring loaded bottom of the storage chamber from being moved towards the metering chamber. The bottom is used for bringing the substance in the reach of the metering chamber. Accordingly, the powder bridges would prevent the substance from being brought into the reach of the metering chamber and, thus, from being finally dispensed to the user. In other words, the formation of powder bridges, would prevent a further setting and dispensing of a dose of the substance. The organizing member is configured for breaking up the powder bridges, thus enabling a continuous and reliable operation of the device. This may help to facilitate provision of an efficient and reliable inhalation device.

A further aspect relates to an inhalation device. The inhalation device may be adapted for the inhalation of a powdery substance. The device may comprise the previously described assembly.

By means of the assembly, in particular by means of the organizing member, formation of powder bridges within the storage chamber may be prevented as described above. Accordingly, a very efficient improved inhalation device may be achieved.

A further aspect relates to the use of an organizing member for agitating a powdery substance. The substance may be retained in a storage chamber of an inhalation device. The inhalation device may be the previously described inhalation device. The organizing member may be shaped ring-like. The organizing member may comprise a main body. The main body may be solid. The organizing member may comprise a cut-out. In particular, the main body of the ring-like organizing member may be broken up due to the cut-out. The angular dimension of the cut-out may be smaller than the angular dimension of the main body.

The organizing member may be adapted and arranged for use within the storage chamber of the device. The solid main body may be used for reducing a volume available within the storage chamber. The cut-out may be used as a symmetry breaker for the organizing member. In particular, due to the specific shape of the organizing member, powdery substance retained in the storage chamber may be agitated upon movement of the organizing member with respect to the storage chamber. Formation of powder bridges within the storage chamber may be prevented.

According to an advantageous embodiment, an assembly for an inhalation device is described. The assembly comprises:

a storage chamber configured to hold a plurality of doses of the substance, and

an organizing member rotatably arranged within the storage chamber for agitating the substance when the organizing member is rotated with respect to the storage chamber.

The organizing member comprises a main body and a cut-out, wherein the main body comprises a solid section of the organizing member, and wherein the angular dimension of the cut-out is smaller than the angular dimension of the main body.

According to an advantageous embodiment, an inhalation device for the inhalation of a powdery substance is described. The device comprises the previously mentioned assembly.

According to an advantageous embodiment, the use of an organizing member for agitating a powdery substance retained in a storage chamber of an inhalation device is described. The organizing member is shaped ring-like. The organizing member comprises a main body and a cut-out, wherein the angular dimension of the cut-out is smaller than the angular dimension of the main body.

Of course, features described above in connection with different aspects and embodiments may be combined with each other and with features described below.

### BRIEF DESCRIPTION OF THE DRAWINGS

Further features and refinements become apparent from the following description of the exemplary embodiments in connection with the accompanying figures.

FIG. 1 schematically shows a sectional side view of an inhalation device,

FIG. 2 schematically shows a sectional side view of a part of an inhalation device,

FIG. 3 schematically shows a perspective side view of an organizing member.

### DETAILED DESCRIPTION

Like elements, elements of the same kind and identically acting elements may be provided with the same reference numerals in the figures.

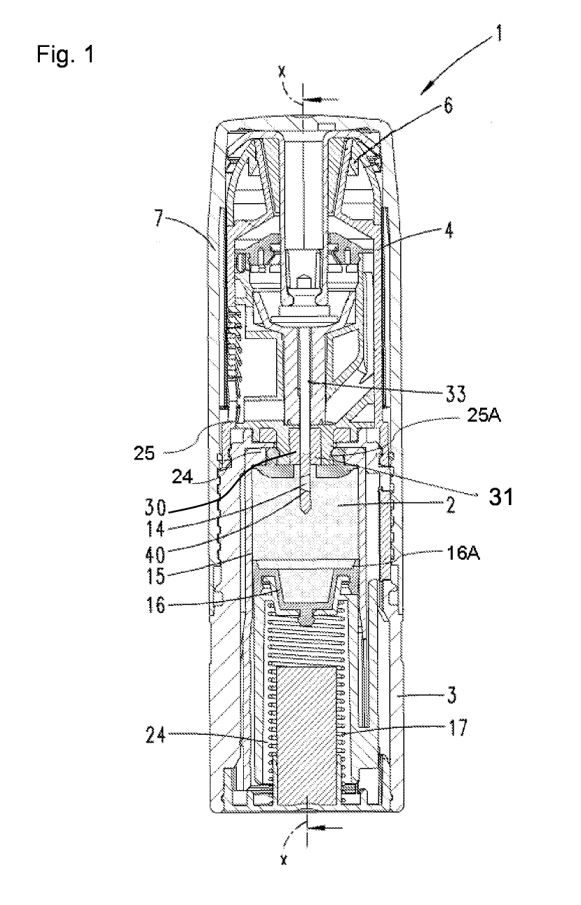

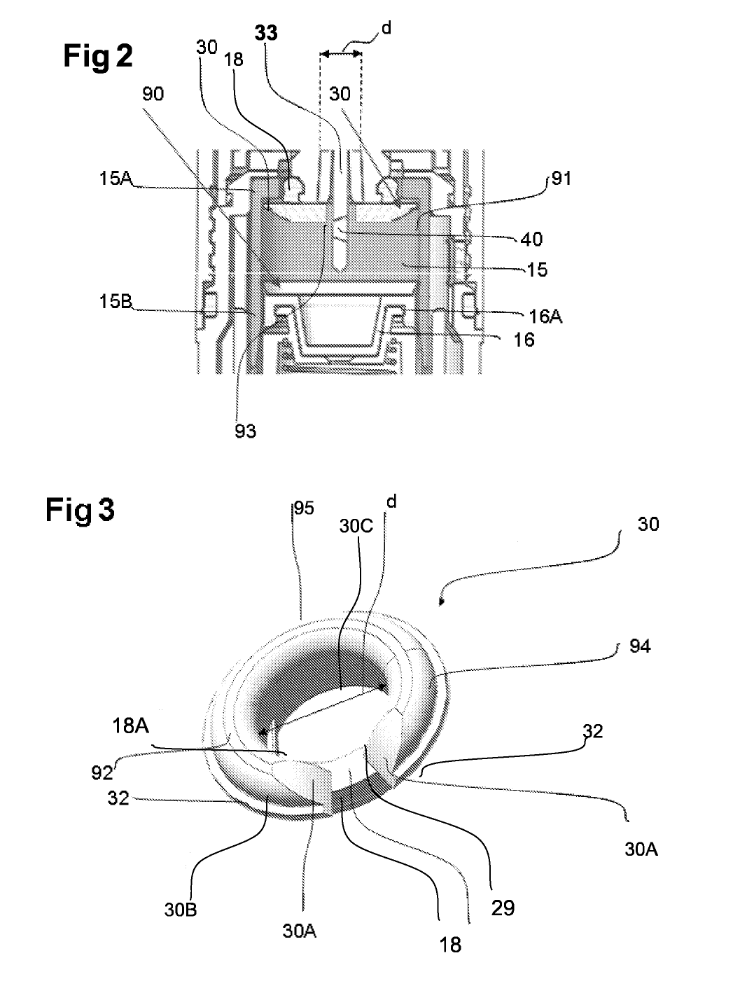

In FIGS. 1 and 2 an inhalation device 1 or parts of the inhalation device 1 are shown. The inhalation device 1 is configured to be actuated by the suction airflow of a user. The inhalation device 1 comprises a housing 3. The device 1 comprises an outer cylinder 4. The outer cylinder 4 is secured against axial movement with respect to the housing 3. The outer cylinder 4 is rotatable with respect to the housing 3.

The inhalation device 1 further comprises a mouthpiece 6. Air is sucked into the device 1 via the mouthpiece 6. The inhalation device 1 comprises a cap 7. The cap 7 is used for covering the mouthpiece 6. The cap 7 comprises a screw cap, for example. The cap 7 may be rotatable with respect to the housing 3 for screwing the cap 7 onto the device 1 and for unscrewing the cap 7 from the device 1. The outer cylinder 4 is rotationally connected, particularly rotationally locked, to the cap 7. In particular, the outer cylinder 4 follows rotation of the cap 7 with respect to the housing 3. For the detailed description of the components of the inhalation device 1 and their mechanical cooperation it is referred to document WO 2009/065707 A1, the entire content of which is explicitly incorporated by reference into the present description.

The device 1 comprises a storage chamber 15. The storage chamber 15 holds one dose, preferably a plurality of doses, of a substance 2. The substance may comprise a drug.

The substance 2 may comprise a powder. The storage chamber 15 comprises a bottom section 15B and a top section 15A. The top section 15A may be the section which is closest to the mouthpiece 6 of the device 1. The bottom section 15B may be the section with is furthest away from the mouthpiece 6.

The inhalation device 1 further comprises a base 16. The storage chamber 15, in particular the bottom section 15B of the storage chamber 15, is terminated by the base 16. The base 16 is formed cup-like. The open end of the cup-like base 16 faces towards the mouthpiece 6 of the device 1. The base 16 comprise an annular lip 16A. The lip 16A may be arranged at the end of the base 16 which is closest to the mouthpiece 6. The lip 16A may comprise an elastomeric material. The lip 16A may be configured to strip substance 2 off an inner wall 91 of the storage chamber 15. The base 16 may seal the storage chamber 15. As substance 2 is removed from the chamber 15 for inhalation by the user, the base 16 may move towards the mouthpiece 6, thereby reducing the volume of the storage chamber 15.

The device 1 further comprises a spring 17. The spring 17 may be a compression spring.

The base 16 is spring-loaded in the direction of the mouthpiece 6 by means of the spring 17 as described in detail in document WO 2009/065707 A1. The spring 17 forces the base 16 in the direction of the mouthpiece 6 when the storage chamber 15 is gradually emptied during operation of the device 1. In other words, due to the spring 17, the base 16 is subjected to a constant spring pressure. In this way, the substance 2 is compressed while the inhalation device 1 is used.

The storage chamber 15, in particular the top section 15A of the storage chamber 15, is terminated by a chamber ceiling 24. The device 1 further comprises a rotary part 25. The rotary part 25 is connected in a rotationally fixed manner to the outer cylinder 4. Accordingly, the rotary part 25 follows rotation of the outer cylinder 4 and, hence, of the cap 7 about the main longitudinal axis x with respect to the storage chamber 15.

The chamber ceiling 24 comprises a central through-opening. A cylindrical portion 25A of the rotary part 25 passes through the central through-opening of the chamber ceiling 24. The lower free end surface of the cylindrical portion 25A is located in the plane of that surface of the chamber ceiling 24 which covers the storage chamber 15. The diameter of the through-opening in the chamber ceiling 26 is larger than the diameter of the cylindrical portion 25A of the rotary part 25.

The device 1 further comprises an organizing member 30. The organizing member 30 is rotationally locked to a part of the device 1 which is rotated along with the cap 7, e.g. the outer cylinder 4. The organizing member 30 is rotationally locked to the cylindrical portion 25, for example. The organizing member 30 is positioned within the storage chamber 15. The organizing member 30 comprises a holder 18 (see also FIG. 3). The holder 18 is part of the organizing member 30. The holder 18 and the organizing member 30 are unitarily formed. The holder 18 is positioned in that end section of the organizing member 30 which is closest to the mouthpiece 6. The holder 18 comprises an annular shape. The holder 18 is positioned in the annular gap which remains between the wall of the through-opening of the ceiling 26 and the outer surface of the cylindrical portion 25A. The organizing member 30 is connected to the cylindrical portion 25A by means of that holder 18. The organizing member 30 may be snap-fitted onto the cylindrical portion 25A. The holder 18 comprises a protrusion 18A (see FIG. 3) for snapping the organizing member 30 to the cylindrical portion 25A. By means of the holder 18, the organizing member 30 is rotationally and axially locked to the cylindrical portion 25A. In other words, the organizing member 30 is secured against axial and rotational movement with respect to the cylindrical portion 25A of the rotary part 25. Thus, the organizing member 30 is rotatable together with the rotary part 25 about the main longitudinal axis x of the device 1. The organizing member 30 is described in more detail in connection with FIG. 3.

The device 1 further comprises a metering rod 33. The metering rod 33 is rotationally locked to the rotary part 25 by mechanical cooperation with the rotary part 25. Accordingly, the metering rod 33 follows rotational movement of the cap 7 and, hence, of the rotary part 25 about the main longitudinal axis x when the cap 7 is mounted onto the device 1 or demounted from the device 1.

The device 1 further comprises a sealing 31. The cylindrical portion 25A accommodates the sealing 31 in its center. The sealing 31 consists of a rubber material or a similar elastic material. The sealing 31 comprises in its center, a slot-like guide opening. The opening is adapted an arranged for receiving the metering rod 33.

The metering rod 33 comprises a metering chamber 40. The metering chamber 40 is positioned offset from the rotation axis of the metering rod 33. The rotation axis is the main longitudinal axis x of the inhalation device 1. The metering rod 33 rotates within the storage chamber 15 before it is fully withdrawn from that chamber 15 in the direction of the mouthpiece 6. For a detailed description of the operation of the metering rod 33, it is referred to document WO 2009/065707 A1. The metering rod 33 is configured for functioning as a moving metering chamber 40 for retrieving a sub-quantity 14 of the substance 2 from the chamber 15, which sub-quantity 14 is to be delivered to the patient. The metering chamber 40 is formed in that end section of the metering rod 33 which projects into the substance 2. The metering chamber 40 is disposed eccentrically in relation to the broad-side surfaces of the metering rod 33. In other words, the metering chamber 40 is offset laterally in relation to the main longitudinal axis x.

When the metering rod 33 and, hence the metering chamber 40, is rotated within the storage chamber 15 for filling the metering chamber 40 with the sub-quantity 14 of the substance 2, the metering rod 33 forms a cavity within the substance 2, for example similar to drilling a hole. The cavity has a shape corresponding to the cross-section of the metering rod 33. In other words, during operation of the metering rod 33, the substance 2 is forced in the radial outward direction towards the inner wall 91 of the storage chamber 15. This radial outward directed force exerted by the metering rod 33 in addition with the force in the direction of the mouthpiece 6 exerted onto the substance 2 by the base 16 may lead to an agglomeration of substance 2, so-called powder bridges, close to the inner wall 91 of the storage chamber 15. In particular, these powder bridges extend along the inner wall 91 of the storage chamber 15. In the worst case, the powder bridges may prevent that the base 16, together with the remaining substance 2, can be pushed in the direction of the mouthpiece 6 during the gradual emptying of the storage chamber 15 while operating the device 1. This would mean that the remaining substance 2 could no longer be pushed into the reach of the metering rod 33 and, thus, substance 2 would no longer be delivered from the device 1. In this case, a large amount of substance 2 would remain unused in the storage chamber 15. In addition, the user could trigger a dose delivery operation without a quantity of the substance 2 being actually dispensed. This may have fatal consequences for the user.

The organizing member 30 is adapted and arranged for avoiding the formation of said powder bridges. In particular, the organizing member 30 is arranged within the storage chamber 15 for agitating the substance 2 when the organizing member 30 is rotated about the main longitudinal axis x with respect to the storage chamber 15. Agitation expediently occurs near the inner wall 91 of the storage chamber 15 and a region which adjoins the region in which the metering rod 33 operates.

FIG. 3 schematically shows a perspective side view of the organizing member.

The organizing member 30 is arranged within the storage chamber 15, which is shown explicitly in FIG. 2, for example. In particular, the organizing member 15 is arranged in the upper end section 15A of the storage chamber 15 as described above. The organizing member 30 is arranged close to the chamber ceiling 24.

The organizing member 30 is shaped ring-like or torus-like. The organizing member 30 comprises an annular shape. The organizing member 30 may comprise a ring, in particular an open ring which is described later on in detail. The organizing member 30 may comprise an oblate or flattened shape. The organizing member 30 comprises a smooth outer shape. In particular, the organizing member 30 comprises smooth chamfers 92. The organizing member 30 comprises a main body 95. The main body 95 is solid. The main body 95 reduces the volume within the storage chamber 15, in particular the volume which is available for the substance 2. The organizing member 30, in particular the main body 95 comprises a cut-out 29. The cut-out 29 is formed as a symmetry-breaker of the main body 95. Substance 2 can be received within the cut-out 29.

The cut-out 29 may run through the whole main body 95 of the organizing member 30. The cut-out 29 may taper-off from an upper end to a lower end of the organizing member 30. The upper end may be the end of the organizing member 30 which is closest to the mouthpiece 6. The lower end may be the end which is furthest away from the mouthpiece 6. Alternatively, the cut-out 29 may comprise an indentation of the main body 95 (not explicitly shown in the Figures). The main body 95 comprises two side faces 30A. The side faces 30A are oppositely arranged with respect to one another. The side faces 30A delimit the cut-out 29 laterally. The side faces 30A are shaped ramp-like. The side faces 30A are oblique with respect to the main longitudinal axis x of the device 1. The side faces 30A comprise rounded, in particular smooth, edges.

The main body 95 of the organizing member 30 comprises an angular dimension, in particular a dimension in the rotation direction of the organizing member 30. The cut-out 29 comprises an angular dimension, in particular a dimension in the rotation direction of the organizing member 30. The angular dimension of the cut-out 29 is smaller than the angular dimension of the main body 95. The angular dimension of the main body 95 may be less than 350°. The angular dimension of the main body 95 may be greater than 270°, preferably greater than 290° or 300°. The angular dimension of the main body 95 may be between 270° and 350°. The angular dimension of the main body 95 may amount to 315°, for example. The angular dimension of the cut-out 29 may be greater than 10°. The angular dimension of the cut-out 29 may be less than 90°, preferably less than 60° or less than 50°. The angular dimension of the cut-out 29 may be between 10° and 90°. Preferably, the angular dimension of the cut-out 29 amounts to 45°, for example.

By means of this specific shape of the organizing member 30 a residual volume 90 which may remain within the storage chamber 15 after the last dose was dispensed may be minimized. When the last dose was dispensed, the organizing member 30 mechanically cooperates with the base 16. In particular, the main body 95 may abut the upper end of the base 16. The shape of the organizing member 30 may be adapted to the shape of the base 16 such that only a minimum space between the base 16 and the organizing member 30 is available for the substance 2 after the last dose was delivered. In this way, the residual amount 90 of the substance 2 remaining in the device 1 is minimized.

The organizing member 30 comprises an outer surface 30B and an inner surface 30C. The organizing member 30 comprises a collar 32. The collar 32 is provided circumferentially around the outer surface 30B of the organizing member 30. The collar 32 protrudes from the outer surface 30B for a distance of 1 mm or less, e.g. of about 0.2 mm. The collar 32 protrudes from the main body 95 such chosen that only a minimal amount of substance 2 can be received between the collar 32 and the inner wall 91.

After the last dose of the substance 2 was dispensed, the collar 32 mechanically cooperates with the base member 16. The main body 95, the collar 32, and the inner wall 91 define a space for receiving the lip 16A of the base member 16. In particular, the collar 32 engages with the lip 16A of the base member 16. In this way, there is no or only a small volume available between the lip 16A and the collar 32 for the substance 2. This may help to further reduce the residual amount 90 of substance 2 remaining within the storage chamber 15 after the last dose was dispensed. All in all, the residual amount 90 of the substance 2 may be reduced by at least 40 sub-quantities 14 of the substance 2 due to the specific shape of the organizing member 30, so as compared to the rotor ring 30 described in document WO 2009/065707 A1. Preferably, the residual amount 90 of the substance 2 may be reduced by 50 sub-quantities 14 of the substance 2 due to the specific shape of the organizing member 30.

The organizing member 30 comprises a central opening 93 (see FIG. 2). The central opening has a diameter d. The diameter d is great enough such that the opening 93 can receive the metering rod 33. In other words, the opening 93 is great enough such that the metering rod 33 can travel through the organizing member 33 for moving the metering chamber 40 into and out of the storage chamber 15. Substance 2 can be received within the central opening 93 of the organizing member 30.

In the following, the operation of the organizing member 30 for agitating the substance 2 is described:

When the cap 7 is mounted onto or demounted from the device 1, the outer cylinder 4 and, hence, the organizing member 30 is rotated about the main longitudinal axis x with respect to the storage chamber 15. When the cap 7 is demounted from the inhalation device 1, the organizing member 30 is rotated in a first direction. When the cap 7 is mounted onto the inhalation device 1, the organizing member 30 is rotated in a second direction with respect to the storage chamber 15. When the organizing member 30 is rotated, the respective side-face 30A is moved, in particular shovelled, through the substance 2. Thereby, the side-face 30A breaks up the substance 2. In particular substance 2 retained in the section close to the inner wall 91 of the storage chamber 15 is broken up by the side-face 30A. In this way, the formation of agglomerations of the substance 2, in particular the formation of powder bridges, is prevented.

When the organizing member 30 is rotated, substance 2 may flow over the smooth, radially inwardly directed edges of the side face 30 in the radial inward direction. Accordingly, the side-face 30A moves substance 2 into the central opening 93 and towards the metering chamber 40. Hence, upon rotation of the metering rod 33, which is rotationally locked to the outer cylinder 4, the metering chamber 40 can be filled with the sub-quantity 14 of the substance 2.

As the organizing member 30 comprises a smooth outer shape, the substance 2 is prevented from being grinded when the organizing member 30 is rotated with respect to the storage chamber 15. Accordingly, an increase of the fraction of fine particles of the substance 2 can be prevented by means of the smooth outer shape of the organizing member 30.

The term drug as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

Other implementations are within the scope of the following claims. Elements of different implementations may be combined to form implementations not specifically described herein.

1-13. (canceled)

14. An assembly for an inhalation device, wherein the assembly comprises:
a storage chamber configured to hold a plurality of doses of a substance,
an organizing member rotatably arranged within the storage chamber for agitating the substance when the organizing member is rotated with respect to the storage chamber,
wherein the organizing member comprises a main body and a cut-out, wherein the main body comprises a solid section of the organizing member, and wherein the angular dimension of the cut-out is smaller than the angular dimension of the main body.

15. The assembly according to claim 14, wherein the organizing member is shaped ring-like.

16. The assembly according to claim 15, wherein the cut-out is delimited by two side faces of the main body, and wherein the side-faces are shaped ramp-like.

17. The assembly according to claim 16, wherein the respective side-face is adapted and arranged to be moved through the substance held in the storage chamber, thereby breaking up substance retained in a section close to an inner wall of the storage chamber when the organizing member is rotated with respect to the storage chamber.

18. The assembly according to claim 14, wherein the main body (95) is adapted and arranged to reduce an inner volume of the storage chamber available for receiving the substance, and wherein the cut-out is configured to receive substance.

19. The assembly according to claim 14, wherein the assembly comprises a base member, and wherein the storage chamber comprises an upper end section and a lower end section, wherein the organizing member is arranged in the upper end section, and wherein the base member is arranged in the lower end section.

20. The assembly according to claim 19, wherein the organizing member comprises a collar, wherein the collar is provided circumferentially along an outer surface of the organizing member.

21. The assembly according to claim 20, wherein the base member is biased in the direction of the upper end section of the storage chamber and wherein the collar is adapted and arranged to mechanically cooperate with the base member.

22. The assembly according to claim 20, wherein the main body, the collar, and the inner wall define a space for receiving a part (16A) of the base member.

23. The assembly according to claim 14, wherein the assembly comprises a metering rod, and wherein the organizing member comprises a central opening which is large enough for the metering rod to travel through the central opening for retrieving a sub-quantity of substance from the storage chamber.

24. The assembly according to claim 14, wherein the organizing member comprises smooth chamfers adapted and arranged to prevent the substance retained in the storage chamber from being grinded when the organizing member is rotated with respect to the storage chamber.

25. Use of an organizing member for agitating a substance retained in a storage chamber of an inhalation device, wherein the organizing member is shaped ring-like, and wherein the organizing member comprises a main body and a cut-out, wherein the angular dimension of the cut-out is smaller than the angular dimension of the main body.

26. Inhalation device for the inhalation of a powdery substance, wherein the device comprises the assembly according to claim 14.
